(19) United States
(12) Patent Application Publication
(10) Pub. No.: US 2013/0289685 A1
(43) Pub. Date: Oct. 31, 2013

(54) IMPLANTATION TOOLS FOR SPINAL CORD STIMULATOR LEADS AND RELATED METHODS

(71) Applicant: NORTH CAROLINA STATE UNIVERSITY, Raleigh, NC (US)

(72) Inventors: Michael Gordon Browne, Whitsett, NC (US); Daniel Joseph Cunningham, Summerfield, NC (US); Meagan Pipes, Clemmons, NC (US); Sudeep Dilip Sunthankar, Columbia, SC (US); John Michael Yanik, Arden, NC (US); Steve Calendar, Raleigh, NC (US)

(73) Assignee: North Carolina State University, Raleigh, NC (US)

(21) Appl. No.: 13/871,022

(22) Filed: Apr. 26, 2013

Related U.S. Application Data
(60) Provisional application No. 61/638,898, filed on Apr. 26, 2012.

(51) Int. Cl. A61N 1/05

(52) U.S. Cl. CPC A61N 1/0553
USPC 607/117

(57) ABSTRACT

Implantation tools for spinal cord stimulator leads and related methods are disclosed. According to an aspect, an implantation tool may include an elongated body having first and second ends. The elongated body may define an interior extending between the first and second ends. The first end may be configured for connection to a vacuum pump for drawing air through the interior in a direction generally from the second end towards the first end. The second end may define an opening for engaging a lead of the spinal cord stimulator when air is drawn by the vacuum pump.

### CROSS REFERENCE TO RELATED APPLICATION

This application claims priority to U.S. Provisional Patent Application No. 61/638,898, filed on Apr. 26, 2012 and titled IMPLANTATION TOOL FOR SPINAL CORD STIMULATOR LEAD, the entire content of which is hereby incorporated by reference.

### TECHNICAL FIELD

The present subject matter relates to the field of medicine. More particularly, the present subject matter relates to implantation tools for spinal cord stimulator leads and related methods.

### BACKGROUND

Spinal cord stimulation (SCS) is used as a treatment for chronic painful conditions. Typically, SCS is used to alleviate pain after failed surgery, pain due to neuropathies, or pain due to inadequate blood flow. SCS theory states that mutual inhibitory connections exist between slow conducting C-fiber sensory neurons and fast conducting non-nociceptive Aβ fiber sensory neurons. It is known that this inhibitory effect can be seen at the level of the second order dorsal horn neurons that project both nociceptive and non-nociceptive sensory information to the brain. Further, it has been recognized that stimulation of non-nociceptive fibers can be used as a therapy to alleviate pain symptoms in cases of chronic pain.

In practice, electrodes are implanted within the epidural space for delivery of electrical stimulation. The electrodes are electrically coupled to a pulse generator which generates high frequency stimulation pulses. More specifically, spinal cord stimulation may apply stimulation pulses to neural tissue of the dorsal column in regular pattern with each pulse being separated by a fixed inter-pulse interval that defines the stimulation frequency. It is believed that high frequency tonic stimulation acts as a “digital lesion” which prevents communication of pain signals to the thalamus of the patient. High frequency stimulation has been observed to prevent the perception of certain types of pain by patients. Instead of perceiving pain, the high frequency electrical stimulation causes other sensation signals to reach the thalamus whereby the patient experiences a tingling sensation known as paresthesia. Although paresthesia can be uncomfortable or even painful to patients, the paresthesia is usually substantially more tolerable than the pain experienced by the patients and hence, is considered an acceptable negative side effect.

Neurosurgeons face the challenge of effectively installing SCS devices. Current SCS installation techniques are tedious and expose the patient to high levels of radiation due to repeated imaging needed for placement of the SCS device. Long surgery can be more harmful to the patient, and it increases both the risk of infection and the cost of a surgery. In view of these shortcomings, it is desired to provide improved techniques and equipment for installing SCS devices.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Disclosed herein are implantation tools for spinal cord stimulator leads and related methods. According to an aspect, an implantation tool may include an elongated body having first and second ends. The elongated body may define an interior extending between the first and second ends. The first end may be configured for connection to a vacuum pump for drawing air through the interior in a direction generally from the second end towards the first end. The second end may define an opening for engaging a lead of the spinal cord stimulator when air is drawn by the vacuum pump.

### BRIEF DESCRIPTION OF THE DRAWINGS

The foregoing summary, as well as the following detailed description of various embodiments, is better understood when read in conjunction with the appended drawings. For the purposes of illustration, there is shown in the drawings exemplary embodiments; however, the presently disclosed subject matter is not limited to the specific methods and instrumentalities disclosed. In the drawings:

FIG. 1 is a perspective view of an implantation tool for a spinal cord stimulator in accordance with embodiments of the present subject matter;

FIG. 2 is a top view of the implantation tool shown in FIG. 1 in accordance with embodiments of the present subject matter;

FIG. 3 is a side view of the implantation tool shown in FIGS. 1 and 2 in accordance with embodiments of the present subject matter;

FIG. 4 is a bottom view of a head portion of the implantation tool shown in FIGS. 1-3 in accordance with embodiments of the present subject matter;

FIG. 5 is a perspective view of an underside of the head portion engaging a lead of a spinal cord stimulator in accordance with embodiments of the present disclosure;

FIGS. 6 and 7 are top and side cross-sectional diagram views of a spine during a surgical procedure for installing a lead of a spinal cord stimulator in accordance with embodiments of the present subject matter;

FIG. 8 is a perspective view depicting example assembly of two pieces of an implantation tool in accordance with embodiments of the present subject matter; and

FIG. 9 is a side perspective view of a portion of an interior of an implantation tool body in accordance with embodiments of the present subject matter.

### DETAILED DESCRIPTION

As used herein, the term “spinal cord” includes any spinal nervous tissue associated with a vertebral level or segment. Those of skill in the art should be aware that the spinal cord and tissue associated therewith are associated with cervical, thoracic, and lumbar vertebrae.

As used herein, the term “epidural space” is known to one of skill in the art, and refers to an area in the interval between the dural sheath and the wall of the spinal canal.

As used herein, the terms “stimulate” or “stimulation” refers to electrical, chemical, heat, and/or magnetic stimulation that modulates predetermined sites in the nervous system.

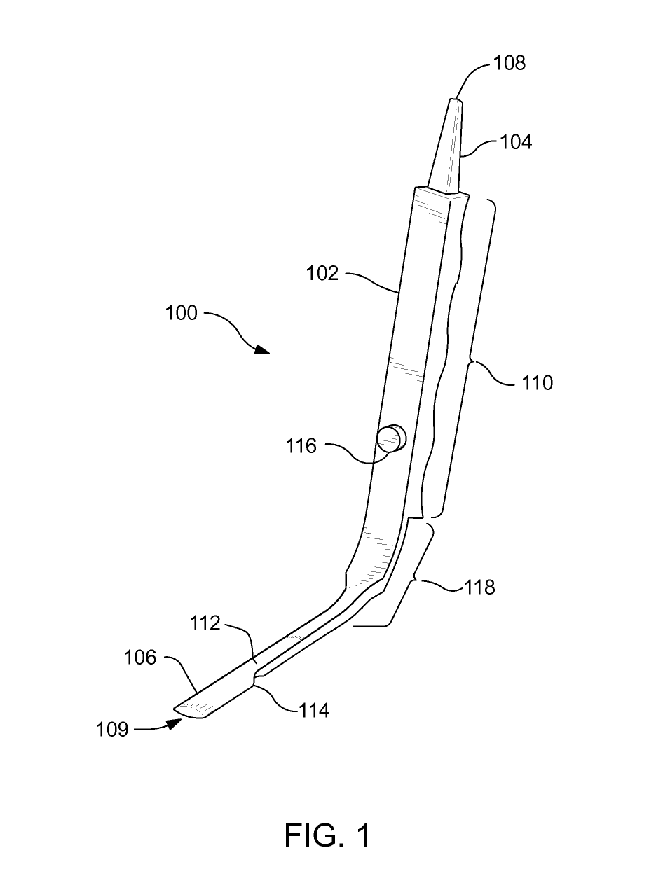

FIG. 1 illustrates a perspective view of an implantation tool, generally designated 100, for a spinal cord stimulator in accordance with embodiments of the present subject matter. Referring to FIG. 1, the tool 100 includes a body 102 having first and second ends 104 and 106, respectively. Although not shown in FIG. 1, the body 102 defines an interior that extends between the first and second ends 104 and 106. As an example, the interior of the body 102 may be substantially open or a narrow passageway may extend between the ends 104 and 106. The interior may allow air to pass freely between the ends 104 and 106.

The first end 104 may be configured for connection to a vacuum pump for drawing air through the interior of the body 102 and in a direction generally from the second end 106 towards the first end 104. More particularly, the first end 104 may include an air hose connector 108 for communicatively connecting the interior of the elongated body to a hose of a vacuum pump. In this example, the air hose connector 108 is conically-shaped and defines an interior passageway leading from the interior of the body 102 to an opening (not shown in FIG. 1) at the end of the connector 108. An air hose of a vacuum pump may be suitably fitted over and attached to the connector 108. The air hose may be attached to connector 108 such that minimal or no air leaks are present at the connection. Alternatively, the air hose connector 108 may have any suitable shape and interior passageway for connecting the tool 100 to a hose of a vacuum pump.

Turning now to the second end 106 of the body 102, this end 106 can define a head portion 109 that is suitably shaped and sized for entry together with a lead of a spinal cord stimulator into the epidural space of a spinal cord. As described in further detail herein, an opening (not shown in FIG. 1) defined within an underside of the second end 106 may lead to the interior of the body 102. The opening may engage the lead of the spinal cord stimulator when air is drawn by the vacuum pump. More particularly, when the opening is placed near the lead and a vacuum pump connected to the first end 104 is activated, the lead may be drawn close to the opening for manipulation of the lead by a surgeon. The surgeon may grip a handle portion 110 of the tool 100 for moving the second end 106 and thereby the lead in a desired direction for placement of the lead in a desired location within the epidural space. Once the lead is in the desired location, the vacuum pump may be turned off, its power suitably reduced, or the air flow through the opening otherwise reduced such that the lead can no longer be manipulated by movement of the end 106.

The handle portion 110 may be suitably shaped and sized for holding by a surgeon. For example, the handle portion 110 may be defined by multiple indentations formed on its exterior surface. The surgeon's fingers may be positioned within the indentations when holding the tool 100.

End 106 may define top and bottom exterior surfaces 112 and 114, respectively. In this example, the distance between the surfaces 112 and 114 is about 2 mm. Alternatively, the distance between the surface 112 and 114 can range between about 1 mm and 3 mm. The width of the end 106 may be any suitable width for permitting movement (e.g., lateral movement) within the spinal column.

The tool 100 may include a valve 116 attached to the body 102 and configured to move between two positions for controlling air flow within the interior of the body 102. As shown in FIG. 1, the valve 116 is attached between the first and second ends 104 and 106, although the valve 116 may be attached at any other suitable position. For example, in one position, the valve 116 may be structured to define a passageway for allowing air to pass between the interior and an exterior of the body 102. In the other position, the valve 116 can partially or fully close the passageway to partially or fully stop the passage of air between the interior and the exterior of the body 102. The flow of air drawn by the vacuum pump through the interior of the body 102 is greater when the valve is in the position to partially or fully stop the passage of air between the interior and exterior of the body 102 as compared to the position to defining the passageway.

In the example of FIG. 1, the valve 116 is attached to the handle portion 110 such that it can be depressed by a surgeon to move between operable positions while the surgeon is holding the handle portion 110. For example, the valve 116 can be positioned on a top surface, as shown in the figure, such that the valve 116 can be depressed and released by the surgeon's thumb. Further, in this example, the valve 116 may include a button shaped and configured to be operated by the surgeon for moving the valve 116 between its various positions.

It is noted that although the valve 116 is provided in the example of FIG. 1, this feature may alternatively be omitted. In alternative embodiments, air flow within the interior of the body 102 may be controlled by the vacuum pump or another suitable technique.

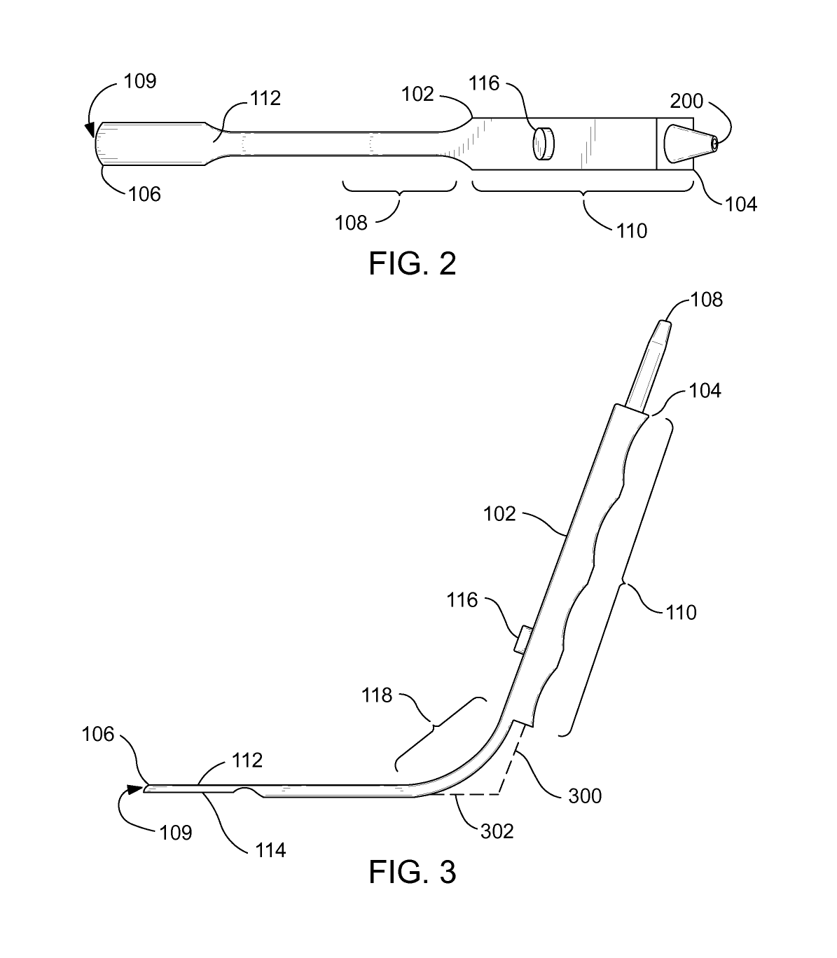

FIG. 2 illustrates a top view of the implantation tool 100 shown in FIG. 1 in accordance with embodiments of the present subject matter. Referring to FIG. 2, an opening 200 of the connector 108 that provides passage from the exterior to the interior of the body 102 is shown.

FIG. 3 illustrates a side view of the implantation tool 100 shown in FIGS. 1 and 2 in accordance with embodiments of the present subject matter. Referring to FIG. 3, broken lines 300 and 302 represent a general direction of respective axes of the handle and head portions 110 and 109, respectively. The broken lines 300 and 302 show that the axes of the handle and head portions are angled with respect to each other. In this example, the angle is about 115 degrees; although the angle can range between about 90 degrees and 150 degrees. Although this example shows a specific angle, it should be recognized by those of skill in the art that the handle and head portions can be angled or otherwise positioned with respect to each other in any suitable way.

In accordance with embodiments of the present subject matter, the body 102 may include a pliable portion including first and second ends connected to handle and head portions, respectively, for allowing the handle and head portions to move with respect to one another. For example, referring to FIGS. 1-3, a pliable portion 118 may be attached to the head portion 109 and the handle portion 110 of the body 102. The pliable portion 118 may be made of a suitable material or suitably shaped such that it flex. In this example, the pliable portion 118 is narrow in comparison to other portions of the body 102 for lateral translation of the head portion 109 when inside the spine for positioning of a lead.

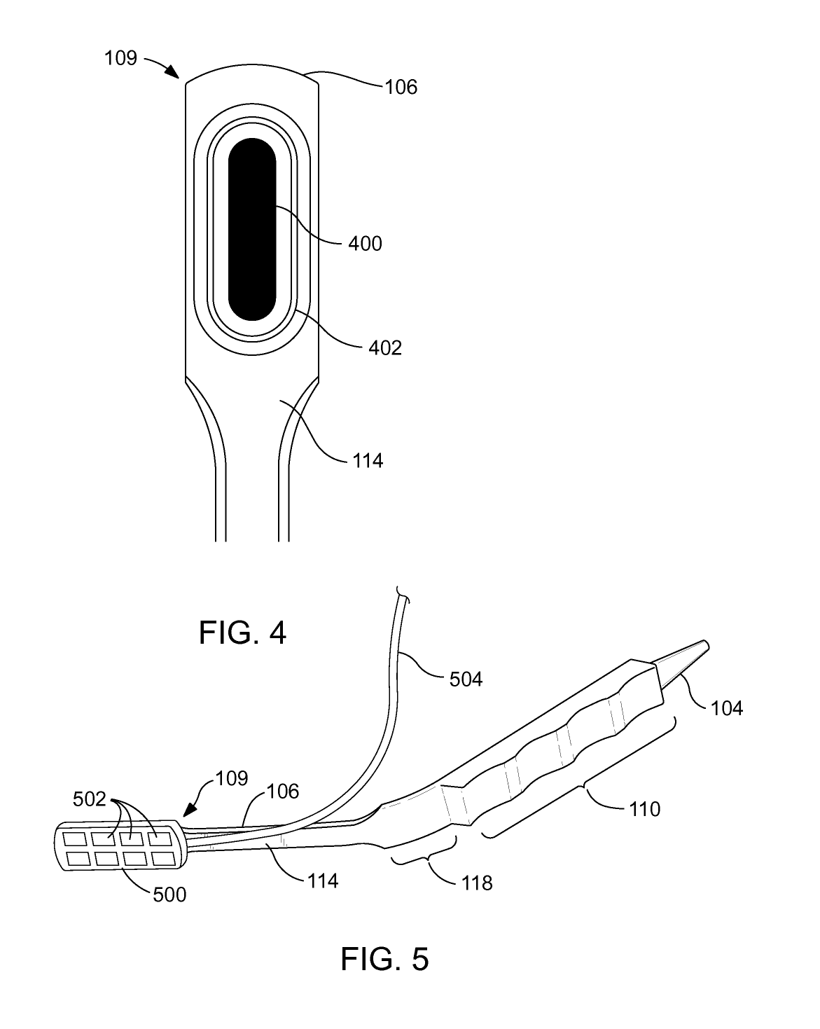

FIG. 4 illustrates a bottom view of the head portion 109 of the implantation tool 100 shown in FIGS. 1-3 in accordance with embodiments of the present subject matter. Referring to FIG. 4, an opening 400 is shown that can engage a lead of a spinal cord stimulator when air is drawn by a vacuum pump connected to the tool 100. In this example, the opening 400 is substantially oval in shape; however, the opening may be any other suitable shape, such as substantially circular. An O-ring 402 may be attached on the bottom exterior surface 114 and encircle the opening 400 as shown. The O-ring 402 may assist with forming a vacuum seal between a lead and the opening 400 when the opening 400 engages the lead.

FIG. 5 illustrates a perspective view of the underside of the head portion 109 engaging a lead 500 of a spinal cord stimulator in accordance with embodiments of the present disclosure. Referring to FIG. 5, the lead 500 may include a plurality of spaced-apart electrodes 502, which are in electrical communication with a pulse generator (not shown) that can generate stimulation pulses. The electrodes 502 may be connected to the pulse generator via an electrical cord 504.

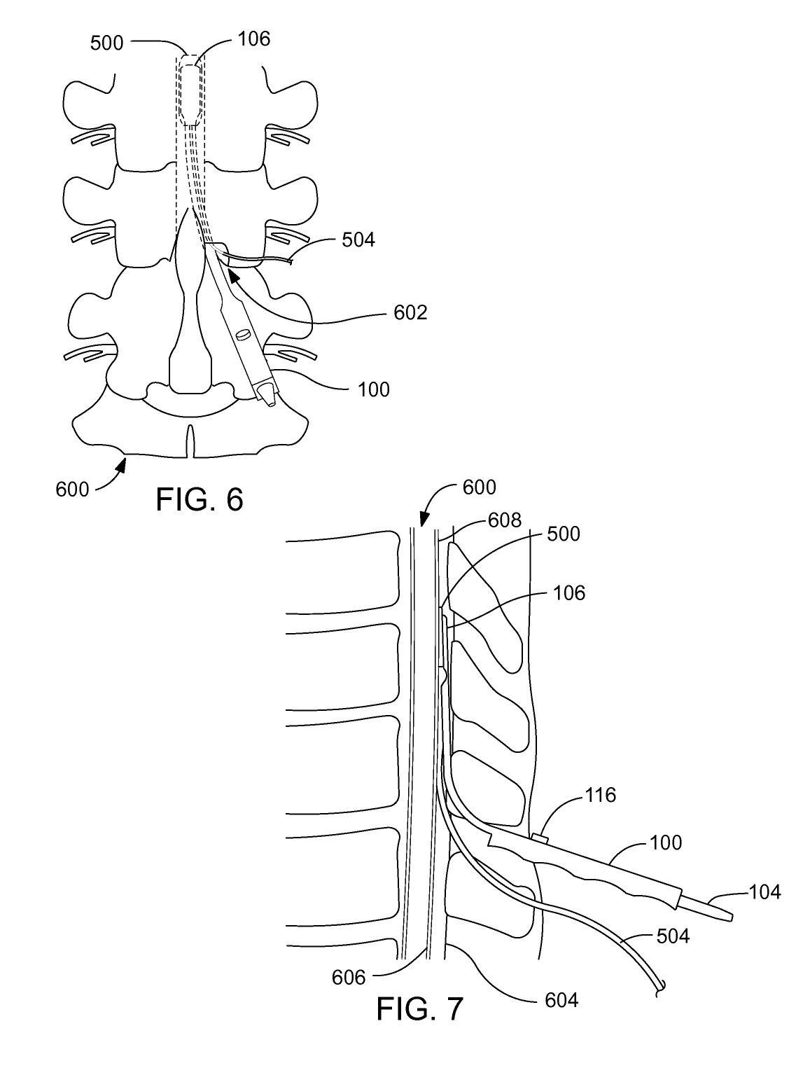

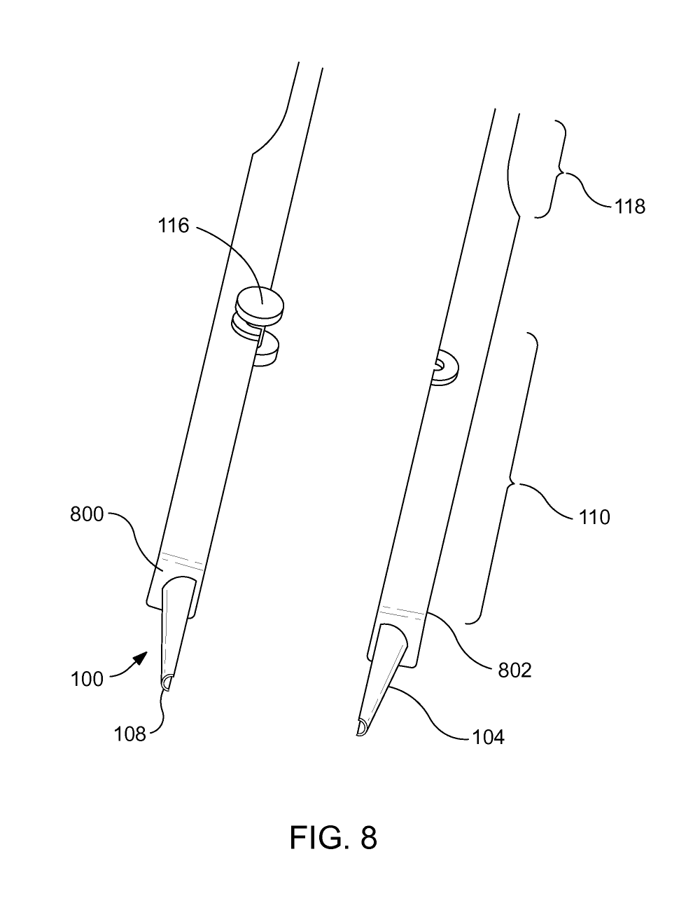

FIGS. 6 and 7 depict top and side cross-sectional diagram views of a spine, generally designated 600, during a surgical procedure for installing a lead 500 of a spinal cord stimulator in accordance with embodiments of the present subject matter. Referring now to FIG. 6, a portion of the spine 600 has been removed via a laminotomy in order to produce an opening 602 large enough for place of the lead 500. The surgical procedure, or partial laminectomy, may require the resection and removal of certain vertebral tissue to allow access to the dura and proper positioning of a lead. An implantation tool 100 is shown being engaged with the lead 500 for its placement within an epidural space 604 of the spine 600. One or more x-ray images may be captured and reviewed to determine whether the lead 500 is positioned as desired. The lead 500 can be implanted such that one or more stimulation electrodes are positioned in communication with the spinal 606. Stimulation electrodes may be placed external to a dura layer 608 surrounding the spinal cord 606. Once the lead 500 is determined to be in a suitable position, the surgeon may release the valve button such that the tool 100 disengages the lead 500. Subsequently, the tool 100 may be removed and surgery completed. Stimulation on the surface of the spinal cord 606 may be applied to the spinal cord tissue as well as to the nerve root entry zone. In examples, a lead may be positioned in various body tissues and in contact with various tissue layers such as subdural, subarachnoid, epidural, and subcutaneous implantation may be employed in some embodiments.

An implantation tool in accordance with embodiments of the present disclosure may be made with any suitable material or material combinations and in any suitable manner. In one or more embodiments, the entirety or a portion of the tool body may be made of an x-ray permissible material such that a lead can be viewed by x-ray imaging without being obscured or blocked by the tool body. As an example, the entire body 102 or only the head portion 109 may be made of x-ray permissible material. In this example, the handle portion 110 may be made of a material that is not x-ray permissible.

In one or more embodiments for tool manufacture, an implantation tool may be manufactured using an acylonite butadiene styrene (ABS) plastic through a process of fusion deposition modeling (FDM). Suitable materials for manufacture may include, but are not limited to, polycarbonate, stainless steel, and medical grade silicone rubber. In an example, the body of the tool may be manufactured in two pieces by a suitable process, such as by use of a 3D fusion deposition modeling printer. After ensuring that an FDM machine is prepared to print and loaded with ABS plastic, the pieces may be fabricated. After printing, pieces may be removed and submerged in a suitable acidic bath to remove structural material that may only be necessary during printing. These pieces may be suitably assembled and a valve attached as shown in FIG. 1.

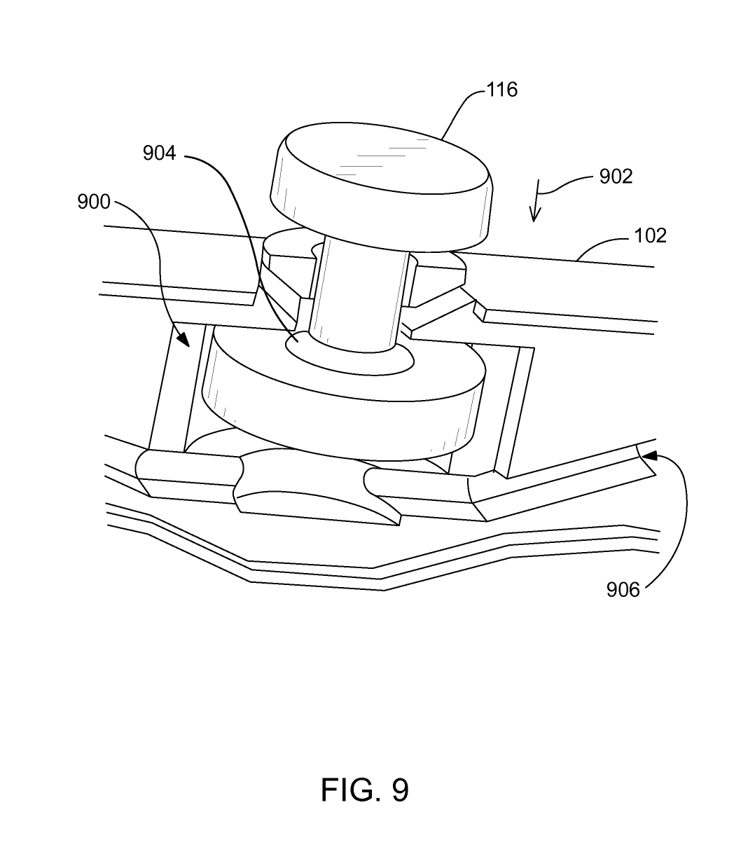

FIG. 8 illustrates a perspective view depicting example assembly of two pieces of an implantation tool 100 in accordance with embodiments of the present subject matter. Referring to FIG. 8, the tool 100 includes two portions 800 and 802 of the body of the tool. The two portions 800 and 802 can be placed together to form the body 102 as shown in FIG. 1. The two portions 800 and 802 may include attachment points such that the portions can fit tightly together. Further, an adhesive may be used to seal the portions 800 and 802 at the attachment points. After the tool has been assembled, the tool may be suitably sterilized for use in a surgical procedure.

FIG. 9 illustrates a side perspective view of a portion of an interior 900 of an implantation tool body 102 in accordance with embodiments of the present subject matter. Referring to FIG. 9, this view shows the portion where a valve 116 is attached to the body 102. The valve 116 may include a spring (not shown) that biases the valve 116 in the position shown in the figure. In this position, the valve closes a passageway to stop passage of air between the interior 900 and the exterior of the body 102. The passageway can form when the valve 116 is depressed in a direction indicated by direction arrow 902. When the valve 116 is in the shown position, an O-ring 904 of the valve 116 engages a surface of the interior 900 to close the airway passage between the interior 900 and the exterior of the body 102. Further, when the valve 116 is in the shown position, air may flow along a passageway 906 that extends between ends of the body 102.

Stimulation of the spinal cord may involve, for example, burst stimulation, which generates bursts of multiple electrical pulses with an inter-burst frequency in the range of about 1 Hz to about 100 Hz, or in the range of about 1 Hz to about 50 Hz. The inter-burst interval may have a duration in the range of about 1 millisecond to about 5 second, or between about 10 milliseconds and about 300 milliseconds. The inter-burst interval need not be constant and can be varied in a programmable manner or varied pseudo-randomly by the pulse generator (e.g., random or irregular harmonics).

The presently disclosed subject matter is described with specificity to meet statutory requirements. However, the description itself is not intended to limit the scope of this patent. Rather, the inventors have contemplated that the claimed subject matter might also be embodied in other ways, to include different elements similar to the ones described in this document, in conjunction with other present or future technologies.

While the embodiments have been described in connection with the various embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed:

1. An implantation tool for a spinal cord stimulator, the tool comprising:
an elongated body comprising first and second ends, and defining an interior extending between the first and second ends, the first end being configured for connection to a vacuum pump for drawing air through the interior in a direction generally from the second end towards the first end, and the second end defining an opening for engaging a lead of the spinal cord stimulator when air is drawn by the vacuum pump.

2. The implantation tool of claim 1, further comprising a valve attached to the elongated body and configured to move between first and second positions, wherein in the first position the valve defines a passageway for allowing air to pass between the interior and an exterior of the elongated body, and wherein in the second position the valve at least partially closes the passageway to at least partially stops passage of air between the interior and the exterior of the elongated body.

3. The implantation tool of claim 2, wherein a flow of air drawn by the vacuum pump through the interior is greater when the valve is in the second position as compared to the first position.

4. The implantation tool of claim 2, wherein the valve is attached between the first and second ends of the elongated body.

5. The implantation tool of claim 2, wherein the body comprises a handle portion.

6. The implantation tool of claim 5, wherein the valve is attached to the handle portion.

7. The implantation tool of claim 2, wherein the valve comprises a button configured to be operated by a user for moving the valve to one of the first and second positions.

8. The implantation tool of claim 1, wherein the body includes an exterior surface, and the body comprises a handle portion defined by a plurality of indentations formed in the exterior surface.

9. The implantation tool of claim 1, wherein the first end of the elongated body comprises an air hose connector for communicatively connecting the interior of the elongated body to the vacuum pump.

10. The implantation tool of claim 1, wherein the body comprises a head portion positioned at the second end.

11. The implantation tool of claim 10, wherein the body comprises a handle portion positioned at the first end.

12. The implantation tool of claim 10, wherein the head portion defines top and bottom exterior surfaces, wherein the opening is defined within the bottom exterior surface.

13. The implantation tool of claim 12, wherein the opening is substantially circular in shape.

14. The implantation tool of claim 12, wherein the opening is substantially oval in shape.

15. The implantation tool of claim 12, further comprising an o-ring that encircles the opening.

16. The implantation tool of claim 1, wherein the body of implantation tool comprises a handle portion and a head portion positioned at the first and second ends, respectively, and wherein axes of the handle and head portions are angled with respect to each other.

17. The implantation tool of claim 1, wherein the body of implantation tool comprises a handle portion and a head portion positioned at the first and second ends, respectively.

18. The implantation tool of claim 17, wherein the body further comprises a pliable portion comprising first and second ends connected to the handle and head portions, respectively, for allowing the handle and head portions to move with respect to one another.

19. A method for implantation of a spinal cord stimulator, the method comprising:
providing an implantation tool comprising an elongated body comprising first and second ends, and defining an interior extending between the first and second ends, wherein the second end defines an opening; and
drawing air through the interior in a direction generally from the second end towards the first end for causing a lead of the spinal cord stimulator to engage the second end when air is drawn.

20. The method of claim 16, further comprising using the implantation tool to implant the spinal cord stimulator within a spinal cord.
